(19) United States
(12) Patent Application Publication
(10) Pub. No.: US 2023/0127505 A1
(43) Pub. Date: Apr. 27, 2023

(54) SYSTEMS AND METHODS FOR PRODUCING MIXTURES

(71) Applicants: Boston Scientific Medical Device Limited, Galway (IE); Boston Scientific Scimed, Inc., Maple Grove, MN (US)

(72) Inventors: Benjamin CLEVELAND, Bellingham, MA (US); Subodh MOREY, Ponda (IN); Nitesh Ghananil BAVISKAR, Kalyan West (IN); Junaid Mohammed SHAIKH, Surat (IN); Richard Earl GRAFFAM, Pelham, NH (US); Joseph HERNANDEZ, Rutland, MA (US); Christopher WATSON, Lincoln, MA (US); Kolbein KOLSTE, Acton, MA (US); Nabarun BHOWMICK, Kolkata (IN); Mihir SUKHATME, Thane (IN); Nachiket GOLE, Pune (IN); Rajivkumar SINGH, Thane (IN); Nicholas DeSANTIS, Cambridge, MA (US); Eric KIM, Brighton, MA (US); Mei Lee AMEND, Lakeville, MA (US)

(73) Assignees: Boston Scientific Medical Device Limited, Galway, MN (IE); Boston Scientific Scimed, Inc., Maple Grove, MN (US)

(21) Appl. No.: 17/970,740

(22) Filed: Oct. 21, 2022

Related U.S. Application Data
(60) Provisional application No. 63/262,937, filed on Oct. 22, 2021.

(51) Int. Cl. A61N 5/10; A61M 5/19

(52) U.S. Cl. CPC A61N 5/1001; A61M 5/19; A61N 2005/1021; A61M 2005/3128

(57) ABSTRACT

A system includes a connector with a central lumen. A multi-lumen chamber is removably connected to and in fluid communication with a proximal end of the central lumen. The multi-lumen chamber includes a first lumen aligned and adjacent a second lumen. The first lumen includes a first fluid in a proximal portion of the first lumen and a hydrophilic polymer in a distal portion of the first lumen, a first plunger rod within the first lumen to control flow of the first fluid into the distal portion to mix with the hydrophilic polymer in a first state to form a first mixture, and a first port. The second lumen includes a second fluid, a second plunger rod within the second lumen to distally move the second fluid and the first mixture in a second state, and a second port.

### CROSS-REFERENCE TO RELATED APPLICATIONS

This patent application claims the benefit of priority under 35 U.S.C. § 119 to U.S. Provisional Patent Application No. 63/262,937, filed Oct. 22, 2021, which is herein incorporated by reference in its entirety.

### FIELD

The present disclosure relates generally to compositions for injection to a patient, methods of preparation and use thereof, and devices comprising such compositions.

### BACKGROUND

Numerous men are diagnosed with prostate cancer each year. Traditionally, treatment options include interstitial implant therapy, surgery, and external beam radiotherapy. While the best treatment is still debatable, side effects of treating prostate cancer have become less toxic with implant therapy and radiotherapy.

Since the conception of conformal radiotherapy, physicians have paid attention to the delivered dose to the target and surrounding tissues. Investigators have been able to correlate side effects to the amount of tissue receiving a certain radiation dose. And yet, time, distance, and shielding affect the dose that is delivered. The less time an area is exposed to radiation, the less dose delivered. The greater the distance from the radiation, the less dose delivered.

Current systems provide filler material to treatment sites to decrease the radiation dose to the rectum during radiotherapy for prostate cancer. However, the system that mixes the filler material in vitro includes numerous subcomponents, is complex to assemble, and rife with filler mixing errors prior to delivery within a patient at a treatment site. During the foregoing procedures, such errors and mishaps lead unnecessarily to patient risk, increased procedure time, and increased procedure costs. The solution of this disclosure resolves these and other issues of the art.

### SUMMARY

In accordance with certain aspects of the present disclosure, a system is disclosed for producing a mixture to deliver to a treatment site. The system can include a mixing lumen including a distal end and a proximal end. A valve can be positioned between the proximal and distal ends. A multi-lumen chamber can be removably connected to and in fluid communication with a proximal end of the mixing lumen with a first lumen aligned and adjacent a second lumen. The first lumen configured to include a first constituent in a proximal portion of the first lumen and a second constituent in a distal portion of the first lumen. A first plunger can be internally positioned within the first lumen to control flow of the first constituent into the distal portion to mix with the second constituent in a first state to form a first mixture. The first lumen can terminate in a first port. The second lumen can be configured to include a third constituent. A second plunger can be internally positioned within the second lumen to distally move the third constituent and the first mixture in a second state. The second lumen can terminate in a second port. Distally moving the second plunger can cause the first mixture and the second constituent to be delivered through the first and second ports and mixed together within the mixing lumen to form the mixture.

In accordance with certain aspects of the present disclosure, the valve is a port extended outward from the mixing lumen with a manually operable valve knob to open and close the port and to prevent backflow from the mixing lumen.

In accordance with certain aspects of the present disclosure, the port further includes a luer fitting configured to receive a distal end of a syringe to deliver a constituent from syringe through the port and the mixing lumen.

In accordance with certain aspects of the present disclosure, the port oriented between approximately 30-90 degrees relative to the mixing lumen.

In accordance with certain aspects of the present disclosure, the valve is a movable stopcock valve configured to control flow through the mixing lumen.

In accordance with certain aspects of the present disclosure, the second plunger can only distally move when the valve is oriented to permit flow through the mixing lumen.

In accordance with certain aspects of the present disclosure, at least one of the first and second lumens includes an external vent in fluid communication with the respective first and second lumen so that air is purgeable through the external vent.

In accordance with certain aspects of the present disclosure, the external vent includes a one-way valve with an air-permeable fluid-impermeable membrane.

In accordance with certain aspects of the present disclosure, air is purged through the external vent only during mixing of the first mixture with the third constituent in the mixing lumen.

In accordance with certain aspects of the present disclosure, the first plunger further includes a proximal flange positioned at a proximal end of the first plunger.

In accordance with certain aspects of the present disclosure, the proximal flange of the first plunger being smaller than a proximal flange of the second plunger.

In accordance with certain aspects of the present disclosure, rotating the proximal flange causes the first plunger to disengage from a distal plunger detachably positioned at a distal end of the first plunger thereby opening a barrier between the proximal and distal portions of the first lumen so that the first constituent mixes with the second constituent to form the first mixture.

In accordance with certain aspects of the present disclosure, moving proximally the proximal flange causes the first plunger to disengage from a distal plunger detachably positioned at a distal end of the first plunger thereby opening a barrier between the proximal and distal portions of the first lumen so that the first constituent mixes with the second constituent to form the first mixture.

In accordance with certain aspects of the present disclosure, proximal and distal ends of the first and second lumens are aligned with each other in the second state.

In accordance with certain aspects of the present disclosure, a needle can be connected to a distal end of the mixing lumen.

In accordance with certain aspects of the present disclosure, a system is disclosed for producing a mixture to deliver to a treatment site. The system can include a mixing lumen including a distal end and a proximal end. A multi-lumen chamber can be connected to and in fluid communication with a proximal end of the mixing lumen with a first lumen aligned and adjacent a second lumen. The first lumen can be configured to include a first constituent in a proximal portion of the first lumen and a second constituent in a distal portion of the first lumen. A first plunger can be internally positioned within the first lumen to control flow of the first constituent into the distal portion to mix with the second constituent in a first state to form a first mixture. The second lumen is configured to include a third constituent. A second plunger can be internally positioned within the second lumen to distally move the third constituent and the first mixture in a second state. Distally moving the second plunger causes the first mixture and the second constituent to be delivered through respective lumen ports and mixed together within the mixing lumen to form the mixture. At least one of the first and second lumens can include an external vent in fluid communication with the respective first and second lumen so that air is purged from the respective first or second lumen through the external vent.

In accordance with certain aspects of the present disclosure, air is purged from the respective first or second lumen through the external vent during mixing of the first mixture and the third constituents to form the mixture.

In accordance with certain aspects of the present disclosure, the external vent includes a one-way valve with an air-permeable fluid-impermeable membrane.

In accordance with certain aspects of the present disclosure, unwanted air of first or second lumen is purged through the external vent by a pressure of fluid flow in the first or second lumen.

In accordance with certain aspects of the present disclosure, after air is purged through the external vent venting, a seal of the external vent is automatically urged to a sealed state thereby preventing flow through the external vent.

In accordance with certain aspects of the present disclosure, a method is disclosed for producing a mixture with a mixing system to deliver to a treatment site. The method can include opening, by a first plunger, a barrier between the proximal and distal portions within the first lumen thereby mixing the first constituent with the second constituent in a first state to form a first mixture; and moving a second plunger causing the first mixture to expel from a first port and the third constituent to expel from a second port and mixed together within the mixing lumen to form the mixture.

In accordance with certain aspects of the present disclosure, a valve is positioned between the proximal and distal ends and is a port extended outward from the mixing lumen. The valve can include a manually operable valve knob to open and close the port and prevent backflow from the mixing lumen.

In accordance with certain aspects of the present disclosure, the method can include connecting a luer fitting of the port further with a distal end of a syringe; and delivering, from the syringe, a constituent through the port and through the mixing lumen.

In accordance with certain aspects of the present disclosure, a valve is positioned between the proximal and distal ends is a movable stopcock valve configured to control flow through the mixing lumen. The second plunger only distally moves when the valve is oriented to permit flow through the mixing lumen.

In accordance with certain aspects of the present disclosure, at least one of the first and second lumens includes an external vent in fluid communication with the respective first and second lumen.

In accordance with certain aspects of the present disclosure, the method can include purging air from the external vent during mixing of the first mixture with the third constituent.

In accordance with certain aspects of the present disclosure, the first plunger includes a proximal flange positioned at a proximal end of the first plunger. The step of opening the barrier includes rotating the proximal flange thereby causing the first plunger to disengage from a distal plunger detachably positioned at a distal end of the first plunger thereby opening the barrier between the proximal and distal portions of the first lumen so that the first constituent mixes with the second constituent to form the first mixture.

In accordance with certain aspects of the present disclosure, the first plunger includes a proximal flange positioned at a proximal end of the first plunger. The step of opening the barrier includes moving proximally the proximal flange thereby causing the first plunger to disengage from a distal plunger detachably positioned at a distal end of the first plunger thereby opening the barrier between the proximal and distal portions of the first lumen so that the first constituent mixes with the second constituent to form the first mixture.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE FIGURES

The accompanying drawings, which are incorporated in and constitute a part of this specification, illustrate various exemplary aspects of the disclosure, and together with the description serve to explain the principles of the present disclosure.

FIGS. 1A-1B depict the prostate, rectum, and Denonvilliers' space between the prostate and rectum.

FIG. 2 shows an upper perspective view of an exemplary mixing system in accordance with certain aspects of the present disclosure.

FIG. 3 shows an exploded view of an exemplary mixing system in accordance with certain aspects of the present disclosure.

FIG. 4A depicts a partial upper plan view of an example step in a method using an example mixing system, in accordance with certain aspects of the present disclosure.

FIG. 4B depicts a partial upper plan view of an example step in a method using an example mixing system, in accordance with certain aspects of the present disclosure.

FIG. 4C depicts a partial side cross-section view of an example step in a method using an example mixing system, in accordance with certain aspects of the present disclosure.

FIG. 4D depicts a partial side cross-section view of an example step in a method using an example mixing system, in accordance with certain aspects of the present disclosure.

FIG. 5 shows close-up perspective view of an exemplary mixing system in accordance with certain aspects of the present disclosure.

FIG. 6A depicts an example step in a method using an example mixing system, in accordance with certain aspects of the present disclosure.

FIG. 6B depicts an example step in a method using an example mixing system, in accordance with certain aspects of the present disclosure.

FIG. 7A depicts an example step in a method using an example mixing system, in accordance with certain aspects of the present disclosure.

FIG. 7B depicts an example step in a method using an example mixing system, in accordance with certain aspects of the present disclosure.

FIG. 8A depicts an example step in a method using an example mixing system, in accordance with certain aspects of the present disclosure.

FIG. 8B depicts an example step in a method using an example mixing system, in accordance with certain aspects of the present disclosure.

FIG. 9A depicts an example step in a method using an example mixing system, in accordance with certain aspects of the present disclosure.

FIG. 9B depicts an example step in a method using an example mixing system, in accordance with certain aspects of the present disclosure.

FIG. 10 depicts an example step in a method using an example mixing system, in accordance with certain aspects of the present disclosure.

FIG. 11 depicts an upper perspective view of an exemplary mixing system in accordance with certain aspects of the present disclosure.

FIG. 12A depicts an upper perspective view of an example step in a method using the example mixing system of FIG. 11, in accordance with certain aspects of the present disclosure.

FIG. 12B depicts a perspective view of an example step in a method using the example mixing system of FIG. 11, in accordance with certain aspects of the present disclosure.

FIG. 12C depicts a perspective view of an example step in a method using the example mixing system of FIG. 11, in accordance with certain aspects of the present disclosure.

FIG. 12D depicts a perspective view of an example step in a method using the example mixing system of FIG. 11, in accordance with certain aspects of the present disclosure.

FIG. 12E depicts a partial, close-up perspective view of an example step in a method using the example mixing system of FIG. 11, in accordance with certain aspects of the present disclosure.

FIG. 13 depicts a flow diagram of a method of using a mixing system according to certain aspects of this disclosure.

### DETAILED DESCRIPTION

Particular aspects of the present disclosure are described in greater detail below. The terms and definitions provided herein control, if in conflict with terms and/or definitions incorporated by reference.

Particular aspects of the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.”

As used herein, the singular forms “a,” “an,” and “the” include plural reference unless the context dictates otherwise.

As used herein, “approximately” and “about” refer to being nearly the same as a referenced number or value. As used herein, the terms “approximately” and “about” should be understood to encompass ±10% of a specified amount or value (e.g., “about 90%” can refer to the range of values from 81% to 99%).

As used herein, “operator” can include a doctor, surgeon, or any other individual or delivery instrumentation associated with delivery or use of a mixing system as such systems are described throughout this disclosure.

The compositions herein may be used in various medical procedures, including but not limited to injected to create additional space between the rectum and prostate during treatment, for example in the Denonvilliers' space, thereby reducing rectal radiation dose and associated side effects. Certain embodiments of the disclosure include placing a filler between the radiation target tissue and other tissues. The filler can be a gel composition that increases the distance between the target tissue and other tissues so that the other tissues receive less radiation.

It is understood that “Denonvilliers' space” is a region located between the rectum and prostate. Certain embodiments provide a method of displacing a tissue to protect the tissue against the effects of a treatment involving radiation or cryotherapy. One embodiment involves using a filler mixed by a mixing system of this disclosure to displace the tissue relative to a tissue that is to receive the treatment. Another embodiment involves introducing a filler mixed by a mixing system of this disclosure to displace a first tissue and radiating a second tissue, particularly a second tissue that is close to the first tissue. In another embodiment, the method includes the steps of injecting a filler into a space between tissues; and may further include irradiating one of the tissues so that the other tissue receives less radiation than it would have in the absence of the filler.

Certain embodiments also provide methods for treating a tissue of a body by radiation. In one embodiment, the method includes the steps of injecting an effective amount of a filler into a space between a first tissue (e.g., prostate) of a body and a second tissue (e.g., rectum), which can be a critically sensitive organ; and treating the first tissue by radiation whereby the filler within the space reduces passage of radiation into the second tissue. Tissue is a broad term that encompasses a portion of a body: for example, a group of cells, a group of cells and interstitial matter, an organ, a portion of an organ, or an anatomical portion of a body, e.g., a rectum, ovary, prostate, nerve, cartilage, bone, brain, or portion thereof.

The gel of the filler can include polymeric materials which are capable of forming a hydrogel may be utilized. In one embodiment, the polymer forms a hydrogel within the body. A hydrogel is defined as a substance formed when an organic polymer (natural or synthetic) is cross-linked via covalent, ionic, or hydrogen bonds to create a three-dimensional open-lattice structure which entraps water molecules to a gel. Naturally occurring and synthetic hydrogel forming polymers, polymer mixtures and copolymers may be utilized as hydrogel precursors.

In some aspects, the hydrogel can be formed by a composition formed by mixing constituents together (E.g., accelerant fluid, diluent, and PEG together) and may comprise one or more polysaccharide compounds or a salt thereof. For example, the composition may include a cellulose compound such as carboxymethyl cellulose (CMC) or salt thereof (e.g., CMC) sodium, xanthan gum, alginate or a salt thereof (e.g., calcium alginate, such as Ca-alginate beads), chitosan, and/or hyaluronic acid. In some examples, the composition may comprise a mixture of hyaluronic acid and CMC, and/or may be cross-linked with a suitable crosslinking compound, such as butanediol diglycidyl ether (BDDE). In some aspects, the polysaccharide may be a homopolysaccharide or a heteropolysaccharide

The present disclosure also provides mixing systems to form the gel composition and corresponding medical devices for use and/or delivery to a treatment site of a patient. According to some aspects of the present disclosure, the mixing system may include a plurality of reservoirs with respective lumens. Collectively, the lumens therein may serve as a container for constituents to mix the gel composition of this disclosure. Suitable reservoirs may include, for example, syringes (e.g., a syringe barrel compatible with a manual or automatic injection system) and other fluid containers configured for use with a suitable injection needle. Exemplary materials suitable for the reservoir include, but are not limited to, cyclic olefin polymer, polypropylene, polycarbonate, polyvinyl chloride, and glass. In some aspects, one of these materials (e.g., cyclic olefin copolymer specifically) can have a coating applied to it, such as SiO₂), which is advantageous so the coating can perform as a primary oxygen barrier, behave as a glass-like layer, and can be applied using a vapor deposition process.

According to some aspects of the present disclosure, the compositions may include at least one accelerant (e.g., an activating agent) combined with a precursor mixed from a diluent (e.g., mostly water) and polyethylene glycol (PEG). In some examples, the composition may be or include a gel with a desired gel strength and/or viscosity, such as a biocompatible gel suitable for injection (e.g., through a needle).

The hydrophilic polymer can be any gelling agent(s), including natural ones or synthetic in origin, and may be anionic, cationic, or neutral. Non-limiting examples of the gelling agents include polysaccharides such as gellan gum, xanthan gum, gum arabic, guar gum, locust bean gum, alginate, and carrageenans.

The concentrations of gelling agent(s) in the composition described in this disclosure may range from about 0.01% to about 2.0% by weight with respect to the total weight of the composition, such as from about 0.02% to about 1.5%, from about 0.05% to about 1.0%, from about 0.05% to about 0.50%, from 0.05% to about 0.15%, from about 0.10% to about 0.20%, from about 0.15% to about 0.25%, from about 0.20% to about 0.30%, from about 0.25% to about 0.35%, from about 0.30% to about 0.40%, from about 0.35% to about 0.45%, from about 0.40% to about 0.50%, from about 0.1% to about 0.5%, or from about 0.1% to about 0.15% by weight with respect to the total weight of the composition. In at least one example, the total concentration of the gelling agent(s) in the composition may range from about 0.05% to about 0.5% by weight with respect to the total weight of the composition.

In some examples, the composition may have a viscosity ranging from about 0.001 Pascal-second (Pa·s) to about 0.100 Pa·s at a shear rate of 130 s⁻¹, such as, e.g., from about 0.005 Pa·s to about 0.050 Pa·s, from about 0.010 Pa·s to about 0.050 Pa·s, from about 0.010 Pa·s to about 0.030 Pa·s, from about 0.010 Pa·s to about 0.020 Pa·s, from about 0.020 Pa·s to about 0.030 Pa·s, or from about 0.020 Pa·s to about 0.040 Pa·s at a shear rate of 130 s⁻¹. Thus, for example, the composition may be or comprise a gel having a viscosity of about 0.005 Pa·s, about 0.006 Pa·s, 0.008 Pa·s, about 0.010 Pa·s, about 0.011 Pa·s, about 0.012 Pa·s, about 0.013 Pa·s, about 0.014 Pa·s, about 0.015 Pa·s, about 0.016 Pa·s, about 0.017 Pa·s, about 0.018 Pa·s, about 0.019 Pa·s, about 0.020 Pa·s, about 0.022 Pa·s, about 0.024 Pa·s, about 0.026 Pa·s, about 0.028 Pa·s, about 0.030 Pa·s, about 0.032 Pa·s, about 0.034 Pa·s, about 0.036 Pa·s, about 0.038 Pa·s, about 0.040 Pa·s, about 0.042 Pa·s, about 0.044 Pa·s, about 0.046 Pa·s, about 0.048 Pa·s, or about 0.050 Pa·s at a shear rate of 130 s⁻¹. In at least one example, the composition may have a viscosity greater than 0.0050 Pa·s at a shear rate of 130 s⁻¹, e.g., a viscosity ranging from about 0.005 Pa·s to about 0.050 Pa·s, at a shear rate of 130 s⁻¹. In at least one example, the composition may have a viscosity greater than 0.010 Pa·s at a shear rate of 130 s⁻¹, e.g., a viscosity ranging from about 0.010 Pa·s to about 0.030 Pa·s, at a shear rate of 130 s⁻¹.

Alternatively or additionally, the composition may have a viscosity ranging from about 0.001 Pa·s to about 0.050 Pa·s at a shear rate of 768 s⁻¹, such as, e.g., from about 0.002 Pa·s to about 0.030 Pa·s, from about 0.003 Pa·s to about 0.020 Pa·s, from about 0.004 Pa·s to about 0.010 Pa·s, from about 0.004 Pa·s to about 0.006 Pa·s, from about 0.005 Pa·s to about 0.007 Pa·s, from about 0.006 Pa·s to about 0.008 Pa·s, from about 0.007 Pa·s to about 0.009 Pa·s, or from about 0.008 Pa·s to about 0.01 Pa·s at a shear rate of 768 s⁻¹. Thus, for example, the composition may be or comprise a gel having a viscosity of about 0.003 Pa·s, about 0.004 Pa·s, about 0.005 Pa·s, about 0.006 Pa·s, about 0.007 Pa·s, about 0.008 Pa·s, about 0.009 Pa·s, or about 0.010 Pa·s at a shear rate of 768 s⁻¹. In at least one example, the composition may have a viscosity less than 0.010 Pa·s at a shear rate of 768 s⁻¹, e.g., a viscosity ranging from about 0.005 Pa·s to about 0.009 Pa·s at a shear rate of 768 s⁻¹. In at least one example, the composition may have a viscosity ranging from about 0.004 Pa·s to about 0.010 Pa·s at a shear rate of 768 s⁻¹. Further, for example, the composition may have a viscosity ranging from about 0.010 Pa·s to about 0.030 Pa·s, e.g., about 0.017 Pa·s at a shear rate of 130 s⁻¹ and a viscosity ranging from about 0.004 Pa·s to about 0.010 Pa·s, e.g., about 0.007 Pa·s, at a shear rate of 768 s⁻¹.

The mixing system herein may include or be removably connected to one or more needles. In some examples, the needle may be a hypodermic needle, and may range from a size of 7 gauge (4.57 mm outer diameter (OD), 3.81 mm inner diameter (ID)) to 33-gauge (0.18 mm OD, 0.08 mm ID), e.g., a size of 16 gauge (1.65 mm OD, 1.19 mm ID), 18 gauge, 21 gauge (0.82 mm OD, 0.51 mm ID), 22 gauge (0.72 mm OD, 0.41 mm ID), 23 gauge (0.64 mm OD, 0.33 ID), or 24 gauge (0.57 mm OD, 0.31 mm ID). Exemplary materials for the needle include, but are not limited to, metals and metal alloys, such as stainless steel and Nitinol, and polymers. The distal tip of the needle may be sharpened, and may have a beveled shape. The proximal end of the needle may include a suitable fitting/adaptor (e.g., a Luer adapter) for engagement with a syringe or other reservoir. In some examples, the needle may include an elongated tube or catheter between the needle tip and the proximal fitting/adapter.

According to some aspects of the present disclosure, the filler compositions herein, e.g., the mixtures and compositions prepared by the methods herein may have sufficient strength, e.g., gel strength, to withstand the forces and thus minimizing the effects of the forces on the continuity of the three-dimensional gel network. In the meantime, the composition with sufficient strength may have a viscosity suitable for injection, e.g., a viscosity that does not render the composition stuck in the reservoir(s), delivery lumen, or a needle connected therewith.

According to some aspects of the present disclosure, the composition may maintain its three-dimensional structure until the gel is injected through a needle, whereupon the structure may form fragments of the original continuous, three-dimensional network. Those gel fragments may have a diameter corresponding to the diameter of the injection needle, such that the fragments are as large as possible in-vivo to retain as much of the three-dimensional structure of the gel as possible. Injection of these larger-sized particles or fragments is believed to increase the amount of time the gel remains within the tissue.

The amount of force required to move the composition through a needle aperture (generally described as “peak load” force) may depend on the viscosity of the composition, the dimensions of the needle (inner diameter, outer diameter, and/or length), and/or the material(s) from which the needle is formed. For example, a greater amount of force may be applied to inject the composition through a 33-gauge needle in comparison to a 7-gauge needle. Additional factors that may affect the amount of force applied to inject the composition may include the dimensions of a catheter (inner diameter, outer diameter, and/or length) connecting the mixing system to the needle. Suitable peak loads for injection with one or two hands may range from about 5 lbf to about 25 lbf, such as from about 10 lbf to about 20 lbf, e.g., about 15 lbf. The loads measured for a given gel concentration may vary for different needles and flow rates.

According to some aspects of the present disclosure, the size of the needle may be chosen based on the viscosity and/or components of the composition, or vice versa. According to some aspects of the present disclosure, the size of the needle may be 23 gauge or 25 gauge. In some cases, a larger size of 18 gauge, 20 gauge, 21 gauge, or 22 gauge may be used to inject the compositions herein.

According to some aspects of the present disclosure, the mixing system of this disclosure can be included in a kit for introducing a filler into a patient, whereby the filler can include any of the gel compositions of this disclosure. Kits or systems for mixing a gel composition of this disclosure, such as hydrogels, may be prepared so that the precursor(s) and any related activating agent(s) are stored in the kit with diluents as may be needed. Applicators may be used in combination with the same. The kits can be manufactured using medically acceptable conditions and contain components that have sterility, purity and preparation that is pharmaceutically acceptable. Solvents/solutions may be provided in the kit or separately. The kit may include syringes and/or needles for mixing and/or delivery. The kit or system may comprise components set forth herein.

During some examples of use, once saline has been injected to the treatment site, a mixing system can be connected to a needle (e.g., an 18 gauge spinal needle) to then inject a 5-10 mm layer of filler (e.g., gel composition) along the posterior wall of the prostate between the prostate and rectum. Once the filler has been injected into the space between the rectum and prostate, ultrasound images can be obtained.

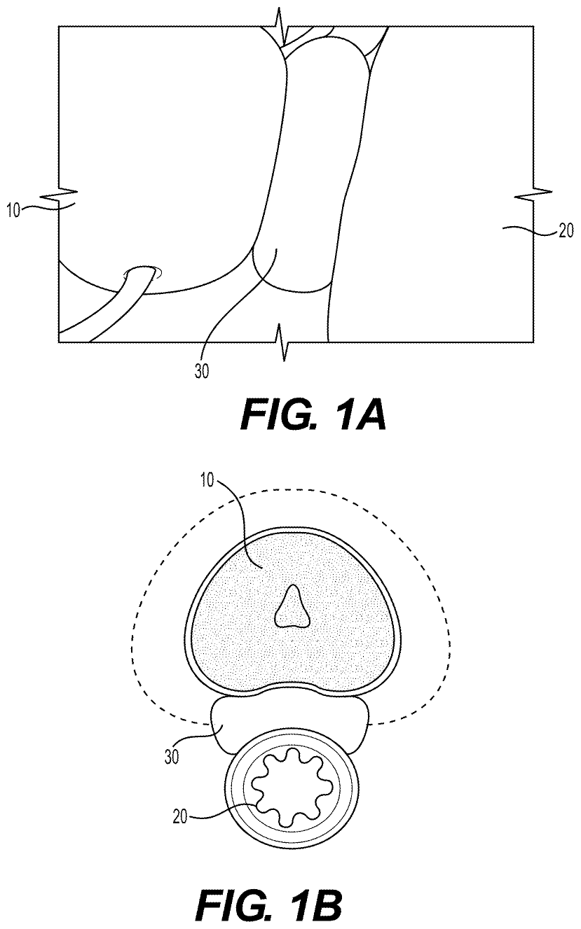

Turning to the drawings, FIG. 1A is a perspective view and FIG. 1B is a partial cross-section view illustrating example filler 30, in the form of a gel composition having been delivered by the mixing system of this disclosure between rectum 20 and prostate 10 of a patient in Denonvilliers' space.

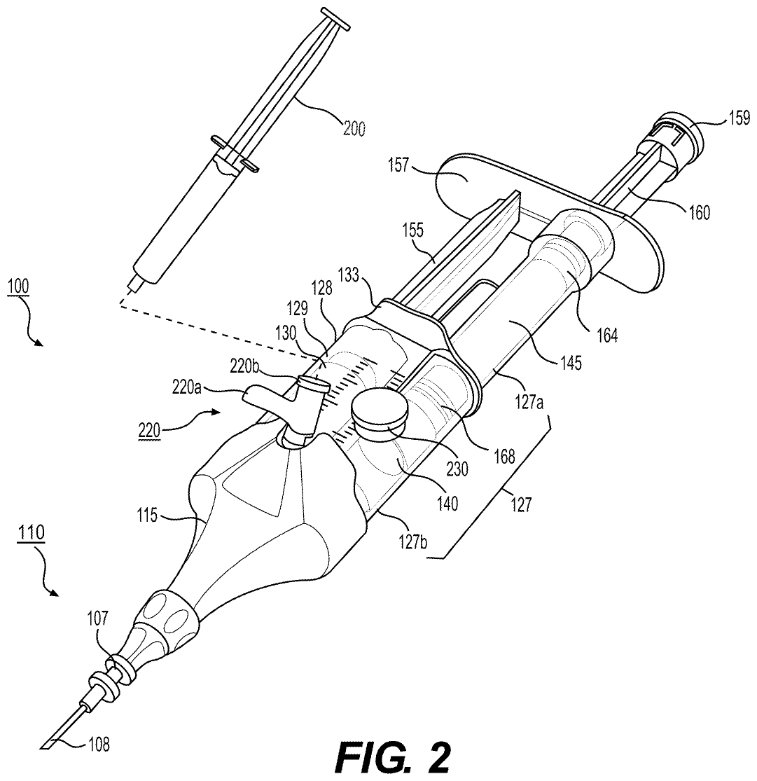

FIG. 2 shows an upper perspective view of an exemplary mixing system 100 in accordance with certain aspects of the present disclosure for mixing a mixture (e.g., a gel composition) for use as filler 30. The system 100 can be packaged in a kit and include a needle assembly 110 attachable therewith, as well as a syringe assembly 200 with a fluid (e.g., saline solution for hydrodissection). Syringe assembly 200 can include a plunger rod with an upper flange surface configured so a user can advance the plunger rod and flush constituent (e.g., saline) from syringe 200 out through fluid port 220b of system 100. Port 220b can be a luer fitting or any other connector operable to engage and be in fluid communication with a distal end of syringe 200.

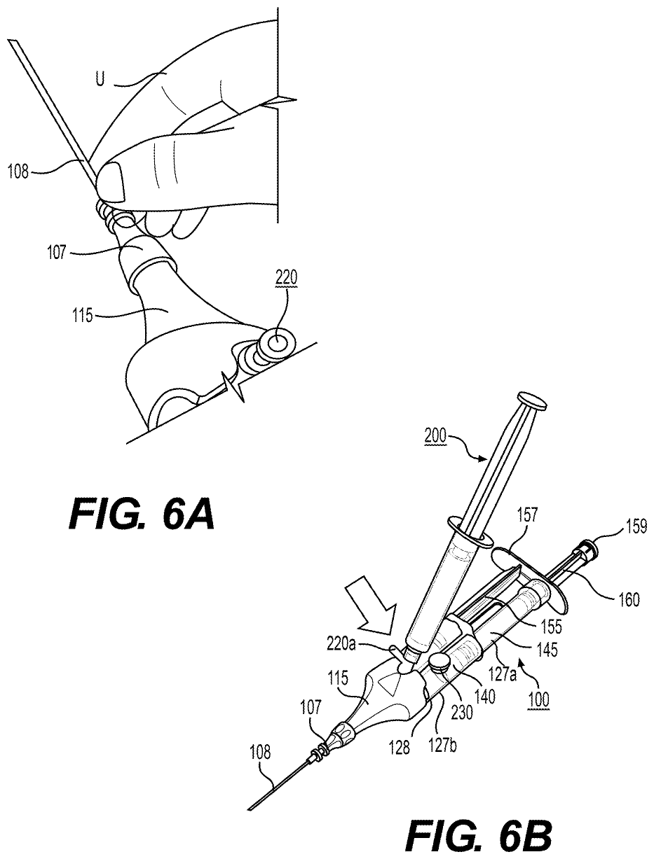

While not shown, during use it is contemplated that needle 108 can be in position at a treatment site of a patient so that filler 30 from system 100 can be delivered to the treatment site. Needle 108 can also be used to deliver saline from syringe 200. After hydrodissection with syringe 200, syringe 200 can be released from fluid port 220b, as clearly shown in FIG. 6B. However, other coupling approaches between connector 115 and syringe 200 are contemplated as needed or required.

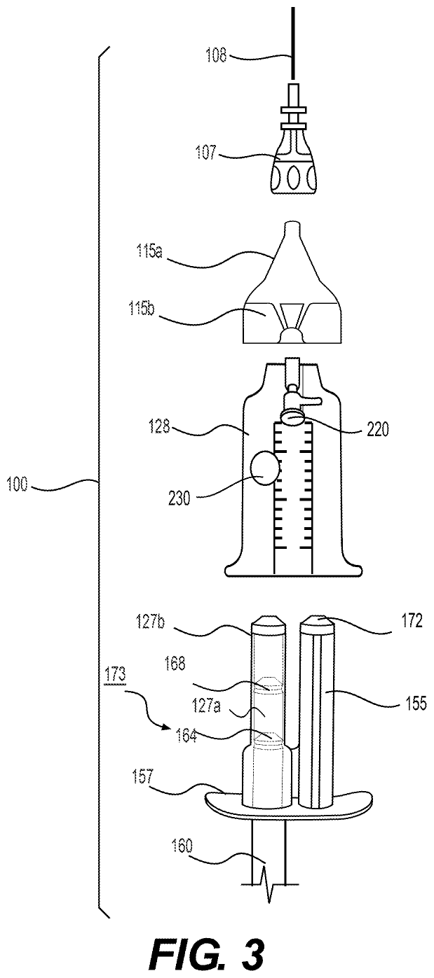

Needle assembly 110 can include needle 108, which can be any needle of this disclosure suitable for hydrodissection as well as to the treatment site delivering filler 30 (e.g., the gel composition). A proximal end of needle 108 can be connected to a distal end of a connector 115. Connector 115 includes a distal portion 115a and a proximal portion 115b, which is shown more clearly in FIG. 3. Portion 115a can be substantially hollow with a tapered or Y-shape profile along its outer surface. Portion 115a can terminate in a distal end with a mixing lumen of connector 115 running therethrough.

System 100 can include a multi-lumen chamber formed by a first lumen 127 inside a first barrel and a second lumen 129 inside a second barrel. Each lumen 127, 129 can be oriented parallel with the other, running side-by-side. Lumen 127 can be divided into a proximal portion 127a and a distal portion 127b. A first plunger stopper 164 located at a distal end of a first plunger rod 160. Rod 160 can be advanceable within lumen 127 and include a first plunger stopper 164 at a distal end of rod 160. Rod 160 can be advanced by button 159 positioned on a proximal end of rod 160.

A second plunger stopper 168 can be positioned within lumen and separate portions 127a, 127b. Portions 127a and 127b can each include constituent(s) (e.g., a fluid, liquid or otherwise). As used herein, the term “fluid” is defined broadly and can include liquids, gels and particulate matter such as granules, pellets, or powders capable of flowing between locations, or any combination of liquids, gels, oils, and/or particulate matter (e.g., granules, pellets, or powders). Distally moving rod 160 can cause stopper 164 to advance constituent(s) of portion 127a so as to open a barrier associated with stopper 168 thereby allowing constituents of each portion 127a, 127b to intermix and form precursor. In some example, constituent 145 of portion 127a can be a diluent fluid solution and portion 127b can include constituent 140 (e.g., an activating agent such as a hydrophilic polymer, PEG or any other agent mixable with diluent to form precursor 145′). The diluent can be a branched polymer having a plurality of succinimidyl termini dissolved in a low pH (4.0) containing a low molecular weight precursor comprising nucleophiles, though other diluent fluid solutions are contemplated within the scope of this disclosure.

Lumen 129 can similarly include a plunger rod 155 slidable therein. A distal end of rod 155 can include a stopper 172. A proximal end of rod 155 can include an actuating flange 157 configured so that a user can a press thereon to drive rod 155 proximally or distally. As seen clearly in FIG. 3, plunger rod 155, flange 157, rod 160, and portions 127a, 127b can be partially or entirely integrally formed together to form plunger assembly 173. System 100 can be assembled to form lumen 129 by positioning distal ends of rods 155, 160 with distal ends of receiver 128. Receiver 128 can include an open proximal end with a flange 133 while the distal ends of receiver 128 can include plurality of smaller openings configured to receive or otherwise provide ports 138 through which constituents of assembly 173 can egress. In some examples, once assembly 173 is assembled with receiver 128, distally moving rod 160 can cause stopper 164 to advance constituent of portion 127a s to open the barrier associated with stopper 168 and allow constituents of each portion 127a, 127b to intermix and form precursor 145′ in portion 127b or distal thereof in a chamber of receiver 128.

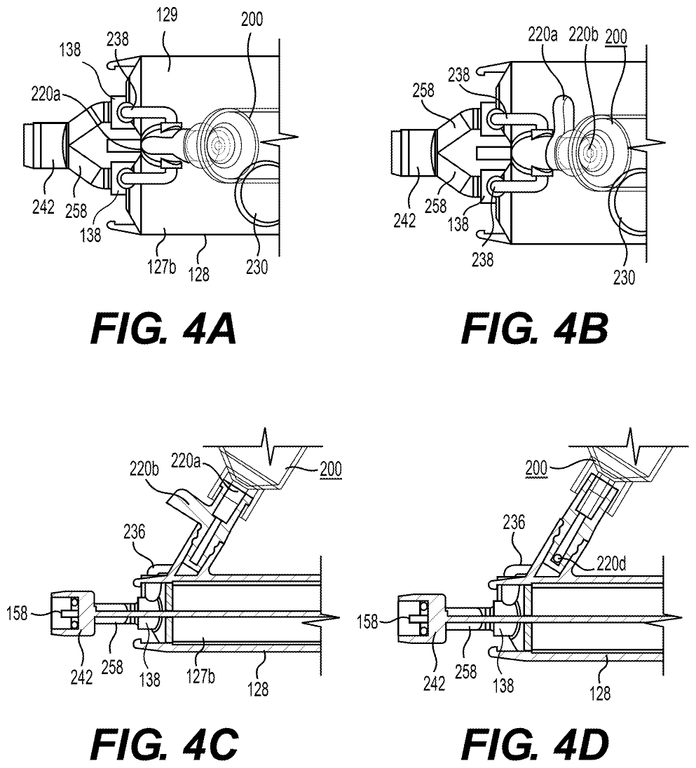

Each of ports 138 are configured to couple to corresponding receivers of connector 115 and permit egress of constituents from respective lumens 127, 129 into connector 115. An example shape and position of ports 138 are clearly shown in FIG. 4A. Portion 115b of connector 115 can be substantially solid with a path running from ports 138 of each lumen 127, 129 to a proximal end of a mixing lumen. The mixing lumen of connector 115 can include a static mixer so that constituent from respective lumens 127, 129 can mix together and form the mixture of filler 30 to be delivered through needle 108. Each of lumens 127, 129 can be in fluid communication with a proximal end of connector 115.

In some aspects, portion 115b of connector 115 can include a tube (e.g., a hypotube) with a proximal end configured in fluid communication with lumen 127 and to pierce a corresponding membrane or seal of port 138. Portion 115b can also include a tube (e.g., a hypotube) with a proximal end configured in fluid communication with lumen 129 and to pierce a corresponding membrane or seal of port 138. In this respect, once precursor 145′ is in position in lumen 127 and constituent 130 is positioned in lumen 129 and connector 115 assembled thereto, distally moving rod 155 can cause precursor 145′ and constituent 130 to egress through respective ports 138 and respective tubes to mix with each other in in the mixing lumen of connector 115. The tubes can form a Y-shape, though any other shape can be used as needed or required.

Optionally, in a first state before mixing, the system 100 can include a retainer removably positioned between flanges 133 and 157 so as to prevent unwanted movement of rod 155. In some aspects, once assembly 173 is nested within receiver 128, lumen 129 is formed between an outer surface of lumen 127 and an inner surface of receiver 128. A constituent 130 (e.g., an accelerant) can be positioned therein, as shown clearly in FIG. 2, and stopper 172 of rod 155 can advance constituent 130 to mix with precursor 145′ once distal of ports 138. In some aspects, while flange 157 is permanently or temporarily attached to button 159 of rod 160, distally advancing flange 157 can distally advance both stopper 172 as well as rod 160, stopper 164, and/or stopper 168 so that the precursor 145′ and constituent 130 are capable of egressing through respective ports 138 and mixing together distal thereof (e.g., in connector 115).

In some aspects, flange 157 can include an opening sized to permit rod 160 to slide therethrough. However, button 159 can be larger than the opening so as to prevent button 159 from sliding distal of flange 157 and ensure that once button 159 and flange 157 are aligned or otherwise attached, flange 157 being distally advanced can drive both rod 160 and rod 155 simultaneously.

System 100 can include a valve 220. Valve 220 can be positioned between proximal and distal ends of connector 115. In some aspects, valve 220 can be a port extended outward from the outer surface of connector 115. Valve 220 can be configured to prevent back flow from connector 115 or any tube or lumen associated with connector 115 or assembly 173. Valve 220 can include a spindle with a valve seat within a respective flow path of connector 115 (e.g., a mixing lumen of connector 115). Valve 220 can include a manually operable valve head 220a extended therefrom and an upper fluid port 220b. As shown, valve 220 can be a movable stopcock valve configured to control flow through the mixing lumen. Port 220b can include a luer fitting configured to receive a distal end of syringe 200 to deliver a constituent (e.g., saline) from syringe 200 through the fluid port 220b and ultimately through needle 108. In some aspects, plunger rod 155 may only be able to distally move when valve 220 is oriented to permit flow through connector 115. Head 220a can be an extension or protrusion from the spindle of valve 220 to facilitate ease of use by user to rotate or otherwise move valve 220 been open and closed positions. Port 220b can be oriented between approximately 30-90 degrees relative to the mixing lumen. Valve 220 can extend from an outer surface of connector 115, as shown in FIG. 2, though it is contemplated that valve 220 can be positioned elsewhere on system 100, including but not limited to extending from an outer surface of at least one of lumens 127, 129.

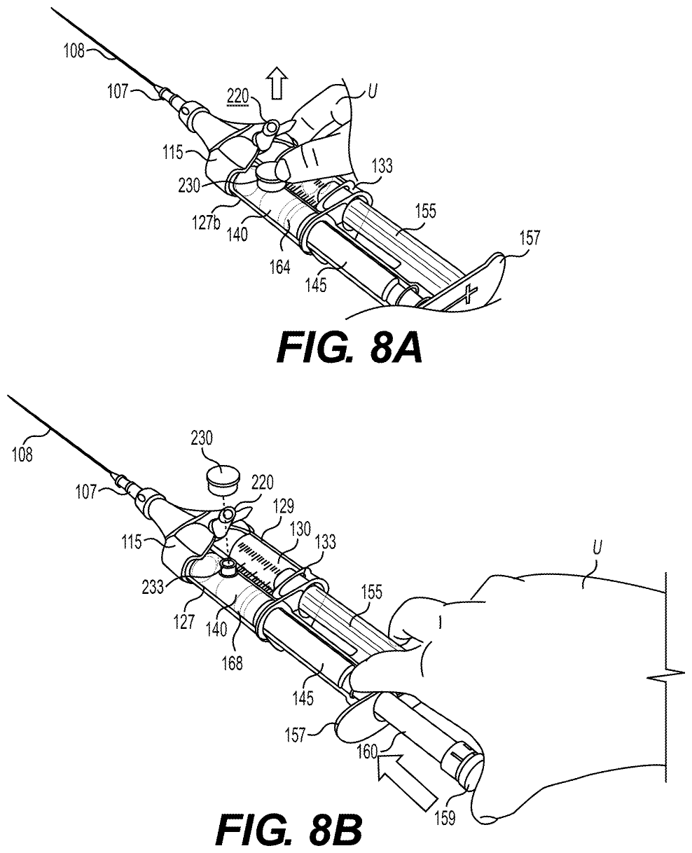

System 100 is particularly advantageous as user can use system 100 to both generate filler 30 (e.g., the gel composition) as well as use the same system for hydrodissection through use of syringe 200 and valve 220, as shown in FIGS. 4A-4D. System 100 can include a detachable cap 230, as illustrated in FIG. 2, so as to seal external vent 233. Vent 233 is shown in the example step of FIG. 8B. Vent 233 can be in fluid communication with lumen 127, but is also contemplated to be in fluid communication and/or positioned on lumen 129. Vent 233 can be configured so that unwanted air can be purged from a respective lumen 127, 129. Vent 233 can include a one-way valve with an air-permeable fluid-impermeable membrane.

In some aspects when vent 233 is in fluid communication with lumen 127 and constituent is urged through receiver 128 or lumen 127, any air distal of constituent in receiver 128 or lumen 127 can be urged through vent 233 by the pressure of the flow. In some examples, vent 233 can include a seal or float that remains in an unsealed state, allowing vent 233 to remain open to discharge air. After initial venting, the seal or float can rise or otherwise be urged to a sealed state with the moving constituent (e.g., precursor 145′) and close vent 233 thereby preventing flow of constituents therethrough.

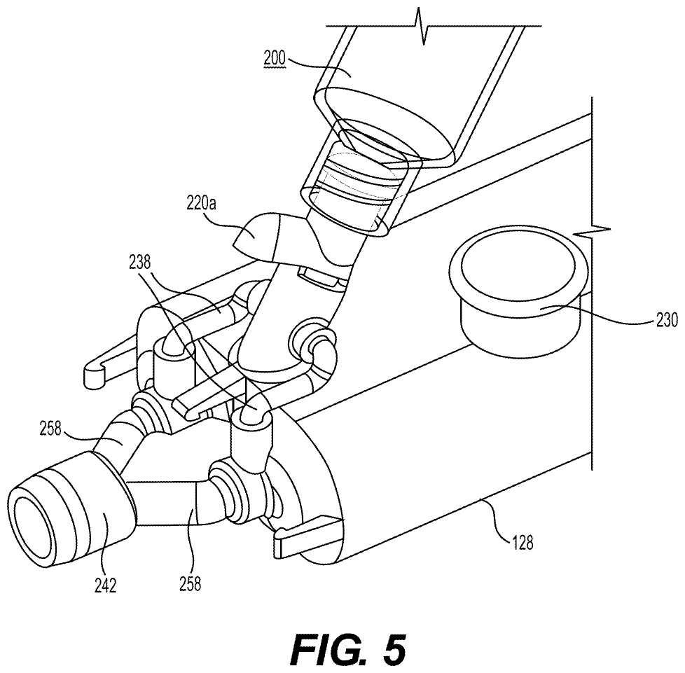

Turning to FIGS. 4A-4D, an exemplary process is shown using valve 220 of system with syringe 200. FIG. 5 shows a close-up perspective view of valve 220 of system 100 in use with syringe 200. Connector 115 is removed from FIGS. 4A-5 strictly to facilitate viewing internal features of system 100. FIG. 4A shows a partial, upper plan view of exemplary syringe 200 in fluid communication and engaged with valve 220, with head 220a positioned aligned with hub 242. When so aligned, this can indicate that valve 220 is opened so constituent can flow therethrough. A fluid path 238 can extend from valve 220 and ultimately to lumens 258 which extend from ports 138 of lumens 127, 129. In FIG. 4B, head 220a has been rotated approximately 90 degrees relative to the valve spindle axis so that the valve 220 is now closed so flow is prevented from flowing therethrough.

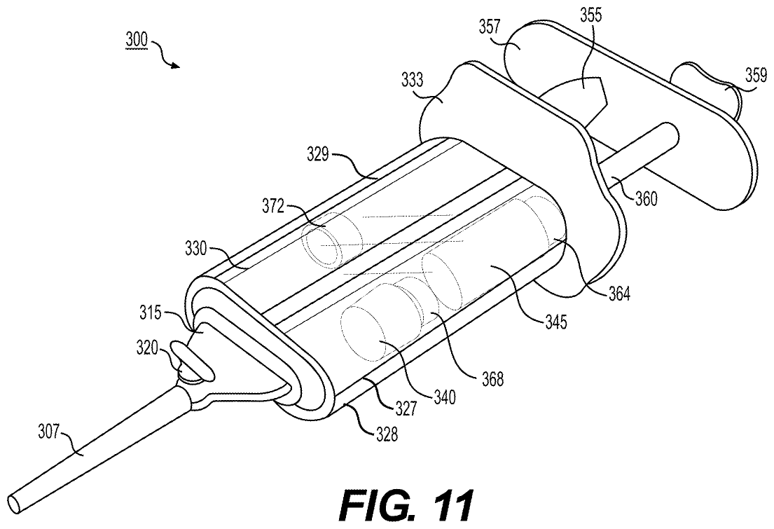

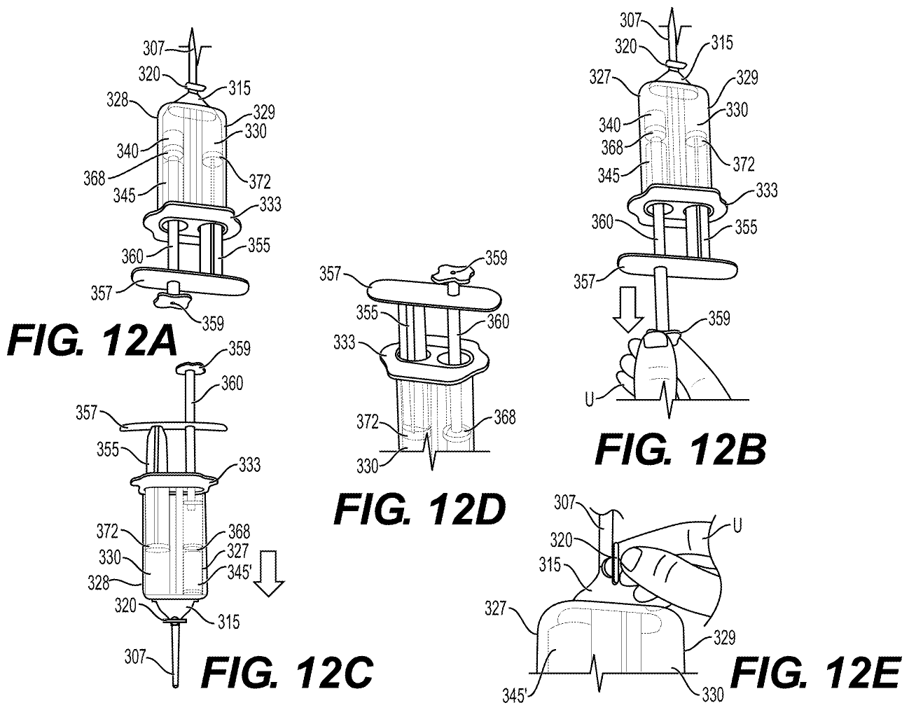

FIG. 4C shows a partial, side cross section view of FIG. 4A, more clearly showing tube 158 extending from lumen 258 through hub 242. FIG. 4D similarly shows a partial-side cross section view of FIG. 4B with valve 220 in the closed position now clearly showing valve seat 220d preventing flow from syringe 200 to system 100. In some examples, it is contemplated that valve seat 220d can be distal of lumens 127, 129, as in FIGS. 11-12E, so as to control flow from each into connector 115.

FIGS. 6A-10B illustrate example steps of a process of using system 100 according to certain aspects of this disclosure. While certain steps are shown as a sequence between each figure, in other embodiments fewer steps are contemplated and the order by which steps are performed can be different than what is illustrated. In FIG. 6A, system 100 is introduced and user attaches needle 108 to connector 115 via needle adaptor 107, which can be on a distal end of connector 115. With needle 108 connected to system 100, in FIG. 6B system 100 is shown connected to syringe 200. Valve head 220a is shown closed with the rod of syringe 200 fully withdrawn to insert saline through port 220b. Rod 160 is also fully withdrawn since constituents 145, 140 have not been intermixed.

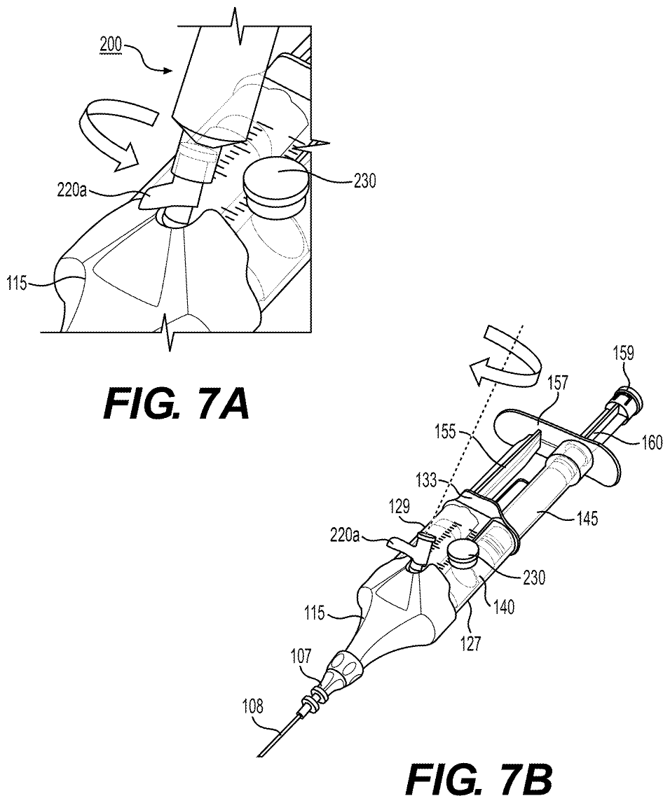

In FIG. 7A, valve head 220a is now moved to be opened and permit flow from syringe 200 into system 100. Now hydrodissection is possible using constituent from syringe 200 through needle 108 and ultimately the treatment site. Once hydrodissection is complete, in FIG. 7B it is shown that syringe 200 can be disconnected from system 100 and valve 220a can again be closed.

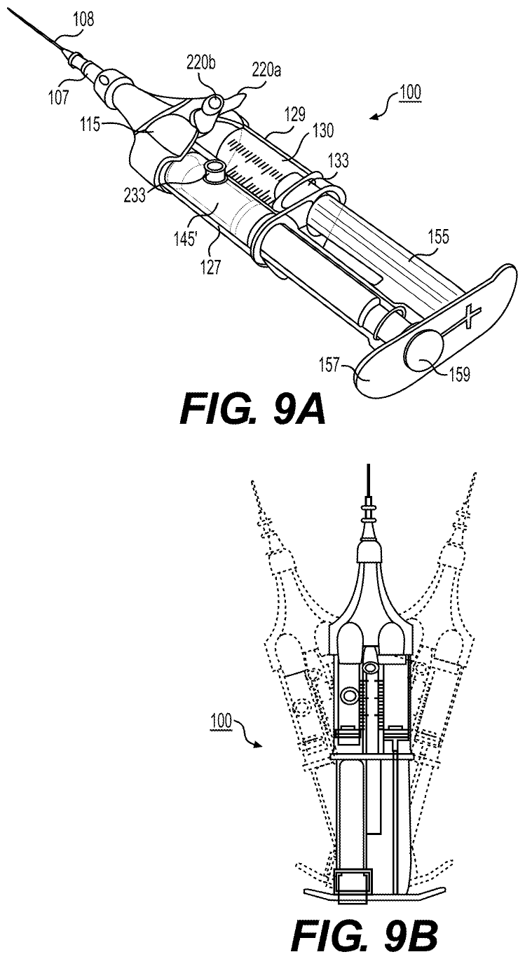

In FIG. 8A, cap 230 can be removed to purge any unwanted air through vent 233. This is seen more clearly in FIG. 8B where cap 230 has been removed. With air purged, rod 160 can be advanced distally by user U causing a barrier of stopper 168 to open so constituents 140, 145 can intermix and form precursor 145′. Rod 160 can be advanced by a user U pressing on button 159 until button 159 is aligned or adjacent flange 157 as in FIG. 9A. In FIG. 9B, system 100 can be shaken back and forth to ensure precursor forms as a result of mixing between constituents 140, 145, while constituent 130 remains in lumen 129. Preferably, the shaking action of FIG. 9B is done while the ports 138 are oriented generally upward. Further, the shaking to effect proper mixing of precursor 145′ can be performed in other orientations (e.g., generally downward, etc.), as needed or required.

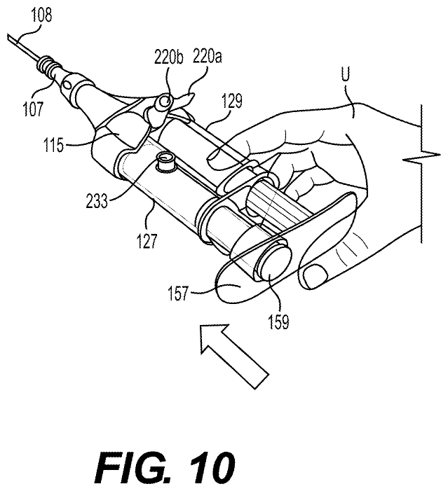

In FIG. 10, with precursor 145′ formed and constituent 130 in lumen 129, flange 157 can now be distally advanced to advance constituents 130, 145′ from respective lumens 127, 129, into connector 115, and ultimately needle 108. With vent 233 uncapped, air A can also be purged as flange 157 is distally advanced. As long as flange 157 is advancing, precursor 145′ and constituent 130 can mix within a mixing lumen of connector 115 and continue egressing through needle 108 and ultimately to the treatment site. Optionally, the connector 115 can include a static mixer configured to thoroughly mix the constituents together to form the gel composition of filler 30 to be delivered to the treatment site. System 100 as shown is relatively easy to assemble and minimizes potential unintentional gel mixing errors prior to delivery.

FIG. 11 shows a perspective view of another exemplary mixing system 300 in accordance with certain aspects of the present disclosure for mixing a gel composition for use as filler 30. Rod 360 and corresponding flange 359 of system 300 is slightly different from previous rod 160 and corresponding button 159. Rather than sliding beyond flange 357, rod 360 can be advanced by flange 359 positioned on a proximal end of rod 360 and is arrange so that flange 359 is incapable and/or prevented from advancing distally past flange 357. In some aspects, an opening of flange 357 through which rod 360 can slide is smaller than the size of flange 359. System 300 can include valve 320, which is similar to previous valve 220, except for valve 320 is located distal of lumens 327, 329 and corresponding port 338 within connector 315 in FIG. 11. In this respect, actuating valve 320 between closed and open positions can control flow of constituents from system 300 and out of connector 315.

FIGS. 12A-12E illustrate example steps of a process of using system 300 according to certain aspects of this disclosure. While certain steps are shown as a sequence between each figure, in other embodiments, fewer steps are contemplated and the order by which steps are performed can be different than what is illustrated. In FIG. 12A, valve 320 is closed thereby preventing flow of constituents from lumens 327, 329 through connector 315 and into adaptor 307. In FIG. 12B, flange 359 can be grasped and rotated by user U so that rod 360 is able to be moved proximally. In FIG. 12 C, with valve 320 still closed, rod 360 can be disengaged from stopper 368 which creates an opening for constituents 345 to intermix with constituent 340 and form precursor 345′. With precursor 345′ formed, in FIG. 12D user U can press flange 359 to distally advance rod 360 to re-engage with stopper 368. Once engaged, in FIG. 12E a user U can now open valve 320 rendering system 300 ready to mix precursor 345′ and constituent 130 in connector 315. Flange 357 can now be distally advanced to advance constituents 330, 345′ from respective lumens 327, 329, into connector 315, and ultimately needle 308.

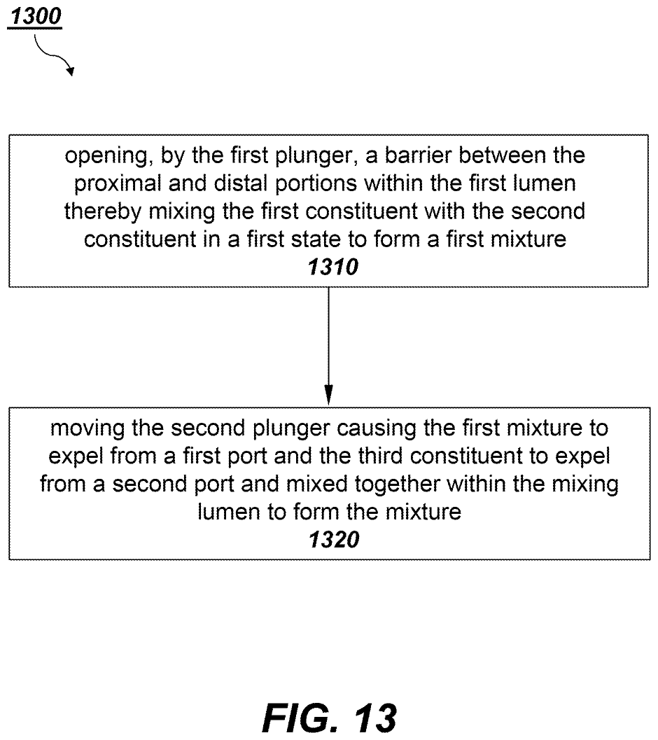

FIG. 13 depicts a method 1300 of using any of the herein disclosed mixing systems. Step 1310 of method 1300 can include opening, by the first plunger, a barrier between the proximal and distal portions within the first lumen thereby mixing the first constituent with the second constituent in a first state to form a first mixture. Step 1320 of method 1300 can include moving the second plunger causing the first mixture to expel from a first port and the third constituent to expel from a second port and mixed together within the mixing lumen to form the mixture. Method 1300 can end after step 1320. In other embodiments, additional steps according to the examples described above can be performed.

The systems and methods of this disclosure are beneficial by reducing the number of system components, are relatively simply to assemble and operate, with minimal mixing errors prior to delivery within a patient at a treatment site. Other aspects and embodiments of the present disclosure will be apparent to those skilled in the art from consideration of the specification and practice of the embodiments disclosed herein.

While certain features of the present disclosure are discussed within the context of exemplary procedures, the compositions, systems, and methods may be used for other medical procedures according to the general principles disclosed. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein. It is intended that the specification and examples be considered as exemplary only, with a true scope and spirit of the present disclosure being indicated by the following claims.

What is claimed is:

1. A system for producing a mixture to deliver to a treatment site, comprising:
a mixing lumen comprising a distal end and a proximal end, and a valve positioned between the proximal and distal ends;
a multi-lumen chamber removably connected to and in fluid communication with a proximal end of the mixing lumen and comprising a first lumen aligned and adjacent a second lumen;
the first lumen configured to comprise a first constituent in a proximal portion of the first lumen and a second constituent in a distal portion of the first lumen, a first plunger internally positioned within the first lumen to control flow of the first constituent into the distal portion to mix with the second constituent in a first state to form a first mixture, the first lumen terminating in a first port; and
the second lumen configured to comprise a third constituent, a second plunger internally positioned within the second lumen to distally move the third constituent and the first mixture in a second state, and the second lumen terminating in a second port;
wherein distally moving the second plunger causes the first mixture and the second constituent to be delivered through the first and second ports, and mixed together within the mixing lumen to form the mixture.

2. The system of claim 1, wherein the valve is a port extended outward from the mixing lumen and comprising a manually operable valve knob to open and close the port and to prevent backflow of fluid from the mixing lumen.

3. The system of claim 2, the port oriented between approximately 30-90 degrees relative to the mixing lumen.

4. The system of claim 1, the first plunger further comprising a proximal flange positioned at a proximal end of the first plunger, the proximal flange of the first plunger being smaller than a proximal flange of the second plunger.

5. The system of claim 1, the first plunger further comprising a proximal flange positioned at a proximal end of the first plunger, wherein rotating the proximal flange causes the first plunger to disengage from a distal plunger detachably positioned at a distal end of the first plunger thereby opening a barrier between the proximal and distal portions of the first lumen so that the first constituent mixes with the second constituent to form the first mixture.

6. The system of claim 1, the first plunger further comprising a proximal flange positioned at a proximal end of the first plunger, wherein moving proximally the proximal flange causes the first plunger to disengage from a distal plunger detachably positioned at a distal end of the first plunger thereby opening a barrier between the proximal and distal portions of the first lumen so that the first constituent mixes with the second constituent to form the first mixture.

7. The system of claim 1, wherein the proximal and distal portions of the first lumen are separated by a barrier; and
wherein distally moving the first plunger causes the barrier to open so the first constituent mixes with the second constituent in the first state to form the first mixture.

8. A system for producing a mixture to deliver to a treatment site, comprising:
a mixing lumen comprising a distal end and a proximal end;
a multi-lumen chamber removably connected to and in fluid communication with a proximal end of the mixing lumen and comprising a first lumen aligned and adjacent a second lumen;
the first lumen configured to comprise a first constituent in a proximal portion of the first lumen and a second constituent in a distal portion of the first lumen, a first plunger internally positioned within the first lumen to control flow of the first constituent into the distal portion to mix with the second constituent in a first state to form a first mixture; and
the second lumen configured to comprise a third constituent, a second plunger internally positioned within the second lumen to distally move the third constituent and the first mixture in a second state;
wherein distally moving the second plunger causes the first mixture and the second constituent to be delivered through respective lumen ports and mixed together within the mixing lumen to form the mixture;
wherein at least one of the first and second lumens comprises an external vent in fluid communication with the respective first and second lumen so that air is purged from the respective first or second lumen through the external vent.

9. The system of claim 8, wherein air is purged from the respective first or second lumen through the external vent during mixing of the first mixture and the third constituents to form the mixture.

10. The system of claim 8, the external vent comprising a one-way valve with an air-permeable fluid-impermeable membrane.

11. The system of claim 8, wherein unwanted air of first or second lumen is purged through the external vent by a pressure of fluid flow in the first or second lumen.

12. The system of claim 8, wherein after air is purged through the external vent venting, a seal of the external vent is automatically urged to a sealed state thereby preventing flow through the external vent.

13. A method for producing a mixture with a mixing system to deliver to a treatment site, the mixing system comprising:
a mixing lumen comprising a distal end and a proximal end;
a multi-lumen chamber removably connected to and in fluid communication with a proximal end of the mixing lumen and comprising a first lumen aligned and adjacent a second lumen;
the first lumen configured to comprise a first constituent in a proximal portion of the first lumen and a second constituent in a distal portion of the first lumen, a first plunger internally positioned within the first lumen to control flow of the first constituent into the distal portion to mix with the second constituent in a first state to form a first mixture; and
the second lumen configured to comprise a third constituent, a second plunger internally positioned within the second lumen to move the second constituent and the first mixture in a second state;
the method comprising:
opening, by the first plunger, a barrier between the proximal and distal portions within the first lumen thereby mixing the first constituent with the second constituent in a first state to form a first mixture; and
moving the second plunger causing the first mixture to expel from a first port and the third constituent to expel from a second port and mixed together within the mixing lumen to form the mixture.

14. The method of claim 13, wherein a valve is positioned between the proximal and distal ends and is a port extended outward from the mixing lumen, the valve comprising a manually operable valve knob to open and close the port and prevent backflow from the mixing lumen.

15. The method of claim 14, further comprising:
connecting a luer fitting of the port further with a distal end of a syringe; and
delivering, from the syringe, a constituent through the port and through the mixing lumen.

16. The method of claim 13, wherein a valve positioned between the proximal and distal ends is a movable stopcock valve configured to control flow through the mixing lumen, and wherein the second plunger only distally moves when the valve is oriented to permit flow through the mixing lumen.

17. The method of claim 13, wherein at least one of the first and second lumens comprises an external vent in fluid communication with the respective first and second lumen.

18. The method of claim 17, further comprising:
purging air from the external vent during mixing of the first mixture with the third constituent.

19. The method of claim 13, the first plunger further comprising a proximal flange positioned at a proximal end of the first plunger, the step of opening the barrier further comprising:
rotating the proximal flange thereby causing the first plunger to disengage from a distal plunger detachably positioned at a distal end of the first plunger thereby opening the barrier between the proximal and distal portions of the first lumen so that the first constituent mixes with the second constituent to form the first mixture.

20. The method of claim 13, the first plunger further comprising a proximal flange positioned at a proximal end of the first plunger, the step of opening the barrier further comprising:
moving proximally the proximal flange thereby causing the first plunger to disengage from a distal plunger detachably positioned at a distal end of the first plunger thereby opening the barrier between the proximal and distal portions of the first lumen so that the first constituent mixes with the second constituent to form the first mixture.
